(19) United States
(12) Patent Application Publication
(10) Pub. No.: US 2023/0048049 A1
(43) Pub. Date: Feb. 16, 2023

(54) METHOD AND SYSTEM FOR DETECTING ONE OR MORE DRUGS AND/OR DRUG METABOLITES IN WASTEWATER

(71) Applicant: Giner, Inc., Newton, MA (US)

(72) Inventors: Avni A. Argun, Newton, MA (US); Muhit Rana, Southborough, MA (US); Badawi M. Dweik, Foxborough, MA (US); Niazul I. Khan, Westmont, IL (US); Andrew Weber, Medford, MA (US)

(21) Appl. No.: 17/887,347

(22) Filed: Aug. 12, 2022

Related U.S. Application Data
(60) Provisional application No. 63/232,463, filed on Aug. 12, 2021.

(51) Int. Cl. G01N 27/06; G01N 33/94; G01N 33/543

(52) U.S. Cl. CPC G01N 27/06; G01N 33/9486; G01N 33/54373

(57) ABSTRACT

Method and system for detecting drug and/or drug metabolites in a liquid sample, such as a wastewater sample. According to one embodiment, the method involves providing a device that includes a graphene field effect transistor and a first aptamer coupled to the graphene field effect transistor in a first well, the first aptamer being selective for a first drug or drug metabolite. Next, a liquid sample is introduced to the first aptamer of the device. Next, a sweeping liquid gate voltage is applied to the device to obtain a resistance versus liquid gate voltage plot for the device. Next, the Dirac voltage shift, if any, in the liquid gate voltage plot for the device is used to determine the presence and/or quantity of the drug or drug metabolite. Additional aptamers selective for different drugs or drug metabolites of interest may also be included in other wells of the device.

### CROSS REFERENCE TO RELATED APPLICATIONS

The present application claims the benefit under 35 U.S.C. 119(e) of U.S. Provisional Pat Application No. 63/232,463, inventors Avni A. Argun et al., filed Aug. 12, 2021, the disclosure of which is incorporated herein by reference in its entirety.

### FEDERALLY SPONSORED RESEARCH OR DEVELOPMENT

This invention was made with government support under 1R43DA051105-01 awarded by the Department of Health and Human Services, National Institute on Drug Abuse. The government has certain rights in the invention.

### BACKGROUND OF THE INVENTION

The present invention relates generally to techniques for detecting drugs and drug metabolites and relates more particularly to techniques for detecting drugs and/or drug metabolites of interest in wastewater and other liquid samples.

The misuse/abuse of drugs including opioids is an epidemic crisis and represents a major threat to public health and safety. The consumption of opioids on a routine basis can cause a variety of adverse health effects, including damage to the central nervous system, and often leads to death. According to a report by the United Nations Office on Drugs and Crime (UNODC), there are almost 300 million people of ages 15 to 64 who use illicit drugs like heroin and pharmaceutical opioids. In 2019 alone, there were more than 70,000 opioid-related deaths in the United States, which corresponds to more than 190 opioid-related deaths per day in the United States. To understand the depth of this crisis, a group of U.S. scientists recently surveyed approximately 3,300 students in ten high schools in the Los Angeles area to learn about the misuse of prescription opioids by high school students. The outcome of this four-year study suggests that 2.1% to 13.1% of students misuse opioids, putting such students at a significant risk for later developing a heroin addiction. Additionally, a recent spike in the presence of highly potent synthetic opioids, such as fentanyl, which is commonly found in street heroin, presents significant concerns and challenges to law enforcement. In order to control illicit drug use and to secure public health and safety, law enforcement needs real-time data on the type and usage frequency of illicit drugs. Current sources for this type of data tend to rely on anonymous surveys or on numbers of cases reported by hospitals or emergency rooms. However, as can be appreciated, such an approach is inadequate as it only reflects a small portion of illicit drug users and does not provide the level of information needed to optimize drug surveillance.

Wastewater-based epidemiology (WBE) (sometimes alternatively referred to as wastewater-based surveillance or wastewater monitoring) is a relatively new approach to drug surveillance and is based on the chemical analysis of target drug analytes in raw wastewater. Wastewater monitoring was first implemented in the U.S. in 2000s and has been utilized primarily in Europe, with several multi-city studies, to monitor illicit drug use. The premise of wastewater monitoring is that, after drug use, drugs are excreted and released into wastewater, mostly in metabolite forms. Wastewater monitoring possesses several advantages over existing surveillance techniques, such as near real-time feedback on usage, the ability to detect changes in daily usage, and information on the introduction of new psychoactive substances. Wastewater monitoring is also able to deliver more rapid, comprehensive, and objective measurement of drug use without stigmatizing individuals or communities.

In order to be successful, however, wastewater monitoring must be very sensitive and specific since such metabolites are typically present in wastewater only at very low concentrations (low pg/mL to ng/mL levels) due to dilution. Unfortunately, at present, only a few methods are available for wastewater monitoring, and none are sensitive and reliable enough to detect low concentrations of opioids in environmental wastewater. The currently preferred technique for wastewater monitoring is high-performance liquid chromatography tandem mass spectrometry (HPLC-MS/MS); however, HPLC-MS/MS often cannot detect opioid metabolites unless the sample is highly processed and the drugs are pre-concentrated. HPLC-MS/MS also needs complex instrumentation and is not amenable for use in wastewater plants, manholes, and catch basins. Field-friendly immunoassays based on antibodies, such as lateral flow immunoassays (LFIs), exist, but such immunoassays exhibit similar degrees of sensitivities to HPLC-MS/MS (1-10 ng/mL), and they are either not quantitative or need complex detectors for analysis. The availability and shelf life of the antibodies used in such immunoassays are also of concern.

### SUMMARY OF THE INVENTION

It is an object of the present invention to provide a novel technique for detecting one or more drugs and/or drug metabolites in wastewater and other liquid samples.

It is another object of the present invention to provide a technique as described above that overcomes at least some of the shortcomings associated with existing techniques.

It is still another object of the present invention to provide a technique that is highly sensitive and specific for detecting one or more drugs and/or drug metabolites of interest and that can be used to analyze a wastewater sample rapidly and accurately. In one embodiment, such a technique may be implemented using a portable, hand-held instrument that may include data storage and wireless communications for real-time dissemination of actionable data.

Therefore, according to one aspect of the invention, there is provided a method for detecting one or more drugs and/or drug metabolites of interest in a liquid sample, the method comprising the steps of (a) providing a device, the device comprising a graphene field effect transistor and a first aptamer coupled to the graphene field effect transistor, the first aptamer being selective for a first drug or drug metabolite of interest; (b) exposing a liquid sample to the first aptamer of the device; (c) then, applying a liquid gate voltage to the device and measuring the resultant resistance; and (d) comparing the resultant resistance to appropriate standards to determine the presence and/or quantity of the first drug or drug metabolite of interest.

In a more detailed feature of the invention, step (c) may comprise sweeping the liquid gate voltage to obtain a resistance versus liquid gate voltage plot for the device.

In a more detailed feature of the invention, step (d) may comprise comparing a Dirac voltage shift for the device to appropriate standards.

In a more detailed feature of the invention, the first drug or drug metabolite may be selected from the group consisting of oxycodone, noroxycodone, fentanyl, norfentanyl, morphine, and 2-ethylidene-1,5-dimethyl-3,3-diphenyl-pyrrolidine.

In a more detailed feature of the invention, the liquid sample may be a wastewater sample.

In a more detailed feature of the invention, the one or more drugs and/or drug metabolites of interest may be exactly one drug or drug metabolite.

According to another aspect of the invention, there is provided a method for detecting one or more drug and/or drug metabolites of interest in a liquid sample, the method comprising the steps of (a) providing a device, the device comprising a graphene field effect transistor, the graphene field effect transistor having a first well and a second well, the device further comprising a first aptamer and a second aptamer, the first aptamer being selective for a first drug or drug metabolite of interest and being coupled to the graphene field effect transistor in a first well, the second aptamer being selective for a second drug or drug metabolite of interest and being coupled to the graphene field effect transistor in a second well, the second drug or drug metabolite of interest being different than the first drug or drug metabolite of interest; (b) exposing a liquid sample to the first aptamer and the second aptamer of the device; (c) then, applying a liquid gate voltage to each of the first well and the second well of the device and measuring the resultant resistance; and (d) comparing the resultant resistance from each of the first well and the second well to appropriate standards to determine the presence and/or quantity of the first drug or drug metabolite of interest and the second drug or drug metabolite of interest.

In a more detailed feature of the invention, step (c) may comprise sweeping the liquid gate voltage in each of the first well and the second well to obtain first and second resistance versus liquid gate voltage plots, respectively, for the device.

In a more detailed feature of the invention, step (d) may comprise comparing a Dirac voltage shift for each of the first and second wells to appropriate standards.

In a more detailed feature of the invention, the one or more drugs and/or drug metabolites of interest may be selected from the group consisting of oxycodone, noroxycodone, fentanyl, norfentanyl, morphine, and 2-ethylidene-1,5-dimethyl-3,3-diphenyl-pyrrolidine.

In a more detailed feature of the invention, the liquid sample may be a wastewater sample.

In a more detailed feature of the invention, the device may further comprise a third well and a third aptamer, the third aptamer may be selective for a third drug or drug metabolite of interest and may be coupled to the graphene field effect transistor in the third well, the third drug or drug metabolite of interest may be different than the first and second drugs or drug metabolites of interest, and the method may further comprise exposing the liquid sample to the third aptamer of the device; then, applying a liquid gate voltage to the third well of the device and measuring the resultant resistance; and comparing the resultant resistance from the third well to appropriate standards to determine the presence and/or quantity of the third drug or drug metabolite of interest.

In a more detailed feature of the invention, the device may further comprise a fourth well and a fourth aptamer, the fourth aptamer may be selective for a fourth drug or drug metabolite of interest and may be coupled to the graphene field effect transistor in the fourth well, the fourth drug or drug metabolite of interest may be different than the first, second and third drugs or drug metabolites of interest, and the method may further comprise exposing the liquid sample to the fourth aptamer of the device; then, applying a liquid gate voltage to the fourth well of the device and measuring the resultant resistance; and comparing the resultant resistance from the fourth well to appropriate standards to determine the presence and/or quantity of the fourth drug or drug metabolite of interest.

According to yet another aspect of the invention, there is provided a device for use in detecting one or more drugs and/or drug metabolites of interest in a liquid sample, the device comprising (a) a graphene field effect transistor, the graphene field effect transistor comprising a first well; and (b) a first aptamer, the first aptamer being coupled to the graphene field effect transistor in the first well, the first aptamer being selective for a first drug or drug metabolite of interest.

In a more detailed feature of the invention, the first aptamer may be selective for a drug or drug metabolite of interest selected from the group consisting of oxycodone, noroxycodone, fentanyl, norfentanyl, morphine, and 2-ethylidene-1,5-dimethyl-3,3-diphenyl-pyrrolidine.

In a more detailed feature of the invention, the first aptamer may be coupled to the graphene field effect transistor using a linker molecule.

In a more detailed feature of the invention, the linker molecule may be pyrenebutyric acid N-hydroxysuccunumide ester.

In a more detailed feature of the invention, the graphene field effect transistor may further comprise a second well, the device may further comprise a second aptamer, the second aptamer may be coupled to the graphene field effect transistor in the second well, the second aptamer may be selective for a second drug or drug metabolite of interest, and the second drug or drug metabolite of interest may be different than the first drug or drug metabolite of interest.

According to a further aspect of the invention, there is provided a system for detecting one or more drugs or drug metabolites in a liquid sample, the system comprising the above-described device, a voltage sweep generator for applying a voltage sweep to the graphene field effect transistor, and a reader/analyzer for measuring the resultant resistance and comparing the resultant resistance to appropriate standards.

Additional objects, as well as aspects, features and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. These drawings are not necessarily drawn to scale, and certain components may have undersized and/or oversized dimensions for purposes of explication or may omit certain features for purposes of clarity. In the drawings wherein like reference numerals represent like parts:

FIG. 1 is a schematic representation of one embodiment of a method for detecting one or more drugs and/or drug metabolites in a wastewater sample according to the present invention;

FIG. 2A is a schematic representation of one embodiment of an aptamer-based graphene field effect transistor for use in the method of FIG. 1, the aptamer-based graphene field effect transistor being constructed according to the present invention and being shown with a drop of liquid containing a drug or drug metabolite of interest;

FIG. 2B is an enlarged fragmentary view of the aptamer-based graphene field effect transistor of FIG. 2A, showing a single graphene field effect transistor with source/drain and side gate electrode and graphene sensing window with A1Oₓ passivation;

FIG. 3A is a top perspective view of a microfluidics cartridge that includes an aptamer-based graphene field effect transistor, the microfluidics cartridge being adapted to receive a liquid sample from a syringe;

FIG. 3B is a top perspective view of one embodiment of a portable reader/analyzer usable with the microfluidics cartridge shown in FIG. 3A, the portable reader/analyzer being shown including a retractable drawer shown in an open position to receive the microfluidics cartridge of FIG. 3A;

FIG. 3C is a top perspective view of the portable reader/analyzer shown in FIG. 3B, with the drawer being shown in a closed position;

FIG. 4A is a schematic representation of the nanoplasmonic assay for aptamer validation that is employed in Example 1, the nanoplasmonic assay utilizing an optical density change resulting from aggregation of gold nanoparticles in the presence of targets;

FIG. 4B is a graph depicting an exemplary change in optical density resulting from performance of the nanoplasmonic assay of FIG. 4A;

FIG. 5A is a graph depicting optical density readings obtained in Example 1 using a noroxycodone aptamer and samples having different noroxycodone levels;

FIG. 5B is a graph depicting the optical density readings obtained in Example 1 using a noroxycodone aptamer and samples containing noroxycodone, norfentanyl, or EDDP, as well as a control sample lacking an opioid;

FIG. 5C is a graph depicting the optical density readings obtained in Example 1 for various wastewater and buffer samples;

FIG. 6A is a graph depicting optical density readings obtained in Example 1 using a norfentanyl aptamer and buffer samples having different norfentanyl levels;

FIG. 6B is a graph depicting optical density readings obtained in Example 1 using an EDDP aptamer and buffer samples having different EDDP levels;

FIG. 6C is a graph depicting optical density readings obtained in Example 1 using wastewater samples containing a norfentanyl aptamer and EDDP, an EDDP aptamer and EDDP, and an EDDP aptamer and norfentanyl, as well as a control with no aptamer or opioid;

FIG. 7A is a schematic top view of the G-FET used in Example 2, showing the active region of the graphene and four-probe contacts;

FIG. 7B is a graph depicting the testing of raw wastewater in Example 2, showing that there was no adverse effect upon exposure to test medium;

FIG. 8A is a graph depicting pyrenebutyric acid N-hydroxysuccunumide ester (PBASE) confirmation by Fourier-transform infrared (FTIR) spectroscopy, as discussed in Example 2;

FIG. 8B is a graph depicting Raman spectra showing D, G and 2D peaks before and after fabrication process, as discussed in Example 2;

FIG. 8C is a graph depicting Raman spectra of graphene over SiO₂/Si substrates before and after aptamer functionalization, as discussed in Example 2;

FIG. 9A is a graph showing some of the specificity confirmation testing discussed in Example 2;

FIG. 9B is a graph showing a calibration curve for noroxycodone in buffer, as discussed in Example 2;

FIG. 10A is a graph showing resistance values obtained as a function of liquid gate voltage for noroxycodone detection in various wastewater samples, as discussed in Example 2;

FIGS. 10B through 10D are calibration curves for noroxycodone, EDDP, and nonfentanyl, respectively, in wastewater samples, as discussed in Example 2; and

FIG. 10E is a graph showing resistance values obtained as a function of liquid gate voltage for noroxycodone detection in various wastewater samples, as discussed in Example 2.

### DETAILED DESCRIPTION OF THE INVENTION

The present invention is directed at a novel technique for detecting one or more drugs and/or drug metabolites of interest in a liquid sample, such as, but not limited to, a wastewater sample. According to one embodiment, the detection technique of the present invention may be achieved using a novel device that comprises (i) a graphene field effect transistor (G-FET) and (ii) one or more aptamers that are coupled to the G-FET, the one or more aptamers being selective or specific for one or more drugs and/or drug metabolites of interest. As will be discussed below, this combination of a graphene field effect transistor and one or more aptamers that are selective or specific for the one or more drugs and/or drug metabolites of interest enables a detection technique that possesses many advantages over existing detection techniques.

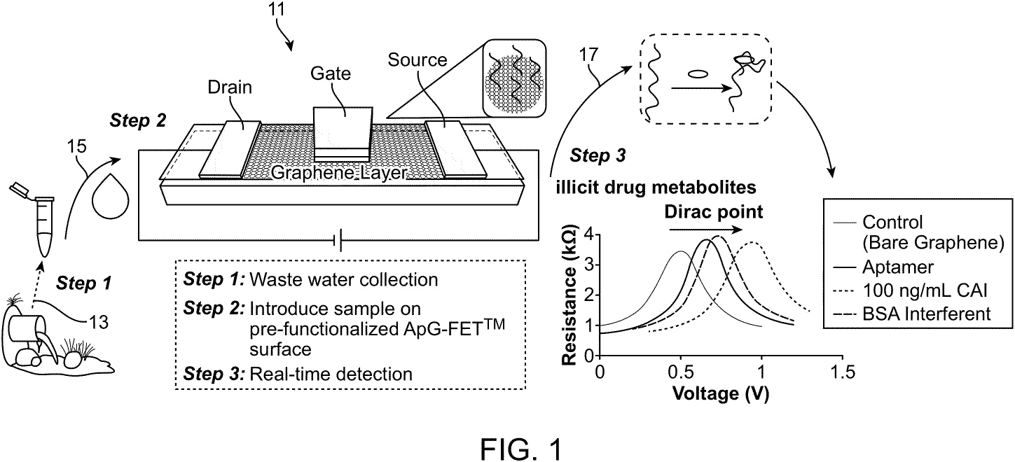

Referring now to FIG. 1, there is shown a schematic diagram of one embodiment of a method for detecting one or more drugs and/or drug metabolites of interest in a liquid sample, the method being represented generally by reference numeral 11. (For simplicity and clarity, certain aspects of method 11 that are not critical to the understanding of the present invention are either not shown or described herein or are shown and/or described herein in a simplified manner.)

Method 11 may begin with a step 13 of collecting a liquid sample. In the present embodiment, step 13 may comprise obtaining a wastewater sample.

Method 11 may continue with a step 15 of introducing the collected sample to a device, the device comprising a graphene field effect transistor (G-FET) to which one or more aptamers that are selective or specific for the one or more drugs and/or drug metabolites of interest have been coupled. If the liquid sample contains the drugs and/or drug metabolites of interest, such metabolites will tend to bind to the aptamers.

Method 11 may continue with a step 17 of detecting the presence of a metabolite that is bound to one of the foregoing aptamers. This may comprise applying a voltage to the G-FET, measuring the resultant resistance, and comparing the resultant resistance to appropriate standards. The resultant resistance may be indicative of the presence of a drug or drug metabolite bound to the aptamer because, when a drug or drug metabolite binds to the aptamer, the additional charge induced on the graphene coupled to the aptamer causes a change in the Dirac point (the Dirac point representing the peak in resistance at charge neutrality). Consequently, by measuring the change in the Dirac point, one not only can determine whether or not the drug or drug metabolite of interest is present but also can determine the concentration of the drug or drug metabolite of interest in the sample.

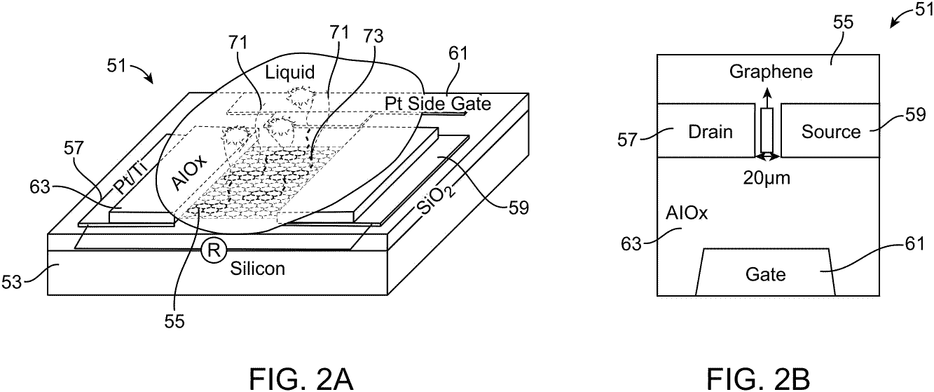

Referring now to FIGS. 2A and 2B, there are shown various views of one embodiment of an aptamer-based graphene field effect transistor of the type that may be used in the method of the present invention, the aptamer-based graphene field effect transistor being constructed according to the present invention and being represented generally by reference numeral 51. (For simplicity and clarity, certain aspects of aptamer-based graphene field effect transistor 51 that are not critical to the understanding of the present invention are either not shown or described herein or are shown and/or described herein in a simplified manner.)

Aptamer-based graphene field effect transistor 51 may comprise a graphene field effect transistor that is similar or identical to one of more of the graphene field effect transistors that are disclosed in the following documents, all of which are incorporated herein by reference: Kumar et al., “Rapid, Multianalyte Detection of Opioid Metabolites in Wastewater,” ACS Nano, 16(3): 3704-3714 (Feb. 24, 2022); Kumar et al., “Detection of a multi-disease biomarker in saliva with graphene field effect transistors,” Med. Devices Sens., 3:e101021 (2020); Kumar et al., “Dielectrophoresis assisted rapid, selective and single cell detection of antibiotic resistant bacteria with G-FETs,” Biosensors and Bioelectronics, 156:112123 (2020); Gray et al., “A Cleanroom in a Glovebox,” Rev. Sci. Instrum., 91(7):073909 (2020).

Accordingly, aptamer-based graphene field effect transistor 51 may comprise a SiO₂/Si substrate 53, a graphene layer 55, a drain electrode 57 (which may be a Pt/Ti electrode), a source electrode 59 (which may be a Pt/Ti electrode), a side gate 61 (which may be Pt), and an A1Oₓ layer 63.

Aptamer-based graphene field effect transistor 51 may further comprise an aptamer 71. Although aptamer-based graphene field effect transistor 51 is shown in FIGS. 2A and 2B as having only a single well, it is to be understood that aptamer-based graphene field effect transistor 51 could include two or more wells. Preferably, many copies of the same aptamer 71 are coupled to graphene layer 55 in a single well, and different types of aptamers may be bound to different wells, with the aptamers of each well being highly selective or specific for a different type of drug or drug metabolite of interest. Such drugs or drug metabolites may be, for example, opioids or opioid metabolites but are not limited thereto.

Aptamers are small, single-stranded nucleic acids (DNA or RNA) that are usually about 20-100 bases in length. Aptamers tend to adopt conformational structures that enable selective binding to a target of interest. To find an aptamer that selectively binds to a target of interest, one may use a technique commonly known as “SELEX,” which typically involves the following steps: (i) start with a large library of aptamers; (ii) immobilize the target; (iii) expose the library of aptamers to the immobilized target; (iv) wash away non-binding aptamers; (v) elute bound aptamers; (vi) amplify the eluted aptamers; (vii) expose the amplified library to the immobilized target; and (viii) repeat steps (iv)-(vii) under conditions of increasing stringency. Additional information relating to aptamers and the SELEX technique for identifying aptamers that are selective or specific for a target of interest may be found in the following documents, all of which are incorporated herein by reference: Zhuo et al., “Recent Advances in SELEX Technology and Aptamer Applications in Biomedicine,” Int. J. Mol. Sci., 18:2142 (2017); Gold, “SELEX: How It Happened and Where It will Go,” J Mol Evol, 81:140-143 (2015); and Ellington et al., “In vitro selection of RNA molecules that bind specific ligands,” Nature, 346:818-822 (1990).

In view of the above, where, for example, the drug or drug metabolite of interest is noroxycodone, one may apply the SELEX technique to a library of aptamers and immobilized noroxycodone to find an aptamer that is selective for noroxycodone. A similar approach may be used to find aptamers that are selective for norfentanyl and EDDP, respectively. In fact, aptamers that are selective for noroxycodone, norfentanyl and EDDP are already commercially available, for example, from Base Pair Biotechnologies, Inc., Pearland, TX, such aptamers including CFA0079-GP5-25, AKA-H4LFD (for binding to noroxycodone); CFA0661-GP5-25 (for binding to EDDP); and CFA0071-GP5-25, AKA-H6AAZ (for binding to norfentanyl). A similar approach may be used to find aptamers selective for other drug metabolites or drugs.

Aptamers 71 may be coupled to graphene layer 55 via a linker molecule 73, such as pyrenebutyric acid N-hydroxysuccunumide ester (PBASE).

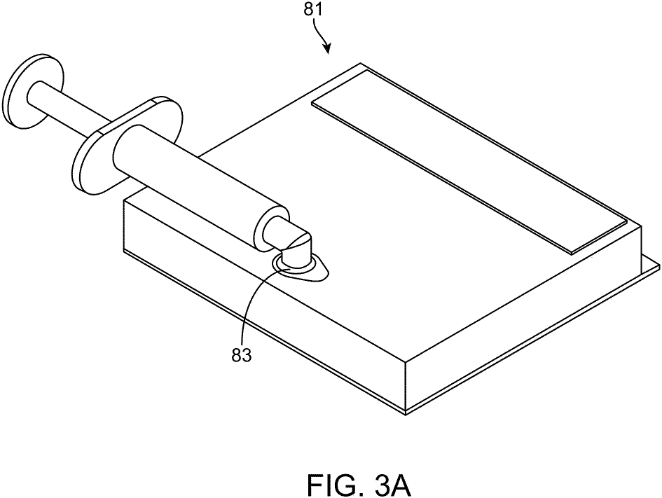

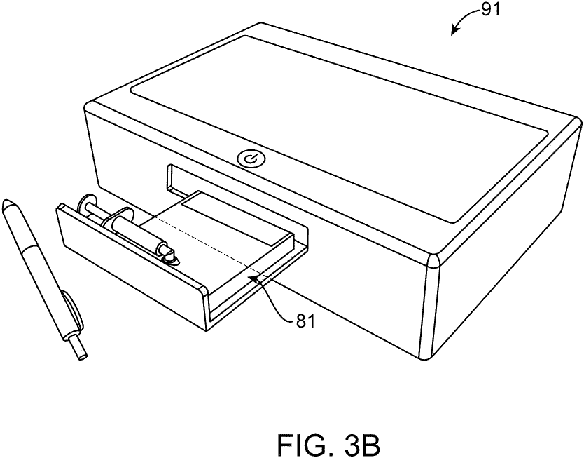

Referring now to FIG. 3A, there is shown a perspective view of a microfluidics cartridge, the microfluidics cartridge being constructed according to the present invention and being represented generally by reference numeral 81. (For simplicity and clarity, certain aspects of microfluidics cartridge 81 that are not critical to the understanding of the present invention are either not shown or described herein or are shown and/or described herein in a simplified manner.)

Microfluidics cartridge 81, which may be designed for multiplexed analysis, may include a port 83 that may be connected to a syringe to receive a liquid sample. Microfluidic cartridge 81 may further include a microfluidic sensor chip (not shown) with a plurality of (e.g., four) independent wells (one drug for each well for simultaneous detection) and a plurality of (e.g., five) G-FET devices per well for signal robustness and reproducibility. Microfluidic cartridge 81 may further include with control interfaces (not shown) which may be operated by a portable analyzer/reader shown in FIGS. 3B and 3C and represented generally by reference numeral 91.

As can be appreciated, the present technique possesses many advantages over existing techniques. Current cutting-edge sensing in wastewater typically relies on high-performance liquid chromatography tandem mass spectrometry (HPLC-MS/MS) or optical techniques, such as enzyme-linked immunosorbent assay (ELISA). These methods achieve high levels of sensitivity and permit the processing of numerous tests in parallel on multiple bioanalytes. However, these methods also require trained personnel and expensive laboratory equipment. As such, collection and testing are rarely done at the same location, with only a limited number of analytes and potential collection sites. Electrical sensors are significantly cheaper, more comfortable to operate, easier for multiplexed analysis, and readily scalable.

Graphene-based sensors offer ultrasensitive, rapid, and accurate detection of targets. The electrical resistance of graphene is highly sensitive to the attached targets (or the probes conformal changes), enabling direct and rapid readout, while the Dirac-point (peak in resistance at charge neutrality) is measured by a gate, providing a quantitative determination of the target concentration. Graphene is also attractive, given its ease of functionalization with an array of biological probes and its ability to be implemented on a wide variety of substrates. Wafer-scale graphene can be commercially purchased from multiple sources in the U.S., Europe, and Asia.

Traditional back-gated FETs require substantial voltages (>60 V) with special electronics. By contrast, the “solution” gated FET sensors of the present invention may employ a side gate directly incorporated onto the chip that operates with up to twenty devices in parallel, offering additional sensitivity, redundancy, and multiplexed detection. Thus, lower voltages (1-2 V) are sufficient for device operation. This is a reliable approach with less complex electronics than are required for other FET approaches. For clean graphene, one observes a maximum in the resistivity at zero applied voltage, indicated as the Dirac Point.

The devices of the present invention can be made by a process such that their intrinsic doping is nearly zero, and the devices are protected by an A1₂O₃ layer, exposing only the active area of graphene and the on-chip electrical gate to the biological targets. This results from a fabrication process that significantly reduces the cost of production and fabrication time. Furthermore, a mask-less lithography system of the type used allows numerous device configurations, and the single-layer graphene on rigid silicon provides a sturdy surface for contact electrodes.

By contrast, recent experimental approaches for drug detection, such as ELISA assays and laminar flow immunoassays, mostly rely on antibodies. Current attempts to multiplex assays using antibodies suffer from inconsistencies between vendors and product lots. The use of aptamers rather than antibodies, coupled with graphene FETs, offer several advantages that enhance their development potential: (1) Aptamers are tolerant to temperatures that cause denaturation of antibodies (no need for refrigeration); (2) Antibody development can take months in animals compared to hours for in vitro aptamer development; (3) Material scales up for aptamers are straightforward via standard in vitro nucleic acid synthesis methods; (4) Functionalization of surfaces with antibodies is not trivial and compromises their binding activity; (5) The strong aptamer binding affinity over antibodies coupled with the high sensitivity of G-FET generates rapid, sensitive, and highly specific biosensors. To turn graphene into a sensor, it may be functionalized with an aptamer specific to the target drug or drug metabolite. When the target binds to the aptamer, the additional charge induced on the graphene is measured by the change in the Dirac point and plotted as a function of the drug or drug metabolite concentration. This enables in-situ optimization of the attachment process. The flat micro-scale, single-layer graphene on a rigid silicon substrate provides a sturdy surface for depositing the contact electrodes by conventional masking techniques.

In summary, there is disclosed herein, according to one embodiment, a compact and label-free graphene field effect transistor sensor utilizing high-specificity aptamers for rapid, sensitive, and multiplexed detection of drugs and/or drug metabolites in wastewater to accurately assess drug misuse and abuse in municipal communities. In particular, in a preferred embodiment, two, three, four or more drugs and/or drug metabolites may be targeted for detection in wastewater samples. For example, such drugs or drug metabolites could include one or more of the following opioids, opioid metabolites or related compounds: oxycodone, noroxycodone (a metabolite of oxycodone), fentanyl, norfentanyl (a metabolite of fentanyl), morphine, and EDDP (a metabolite of methadone, fully synthetic opioid. The strong opioid metabolite/aptamer binding complex at the graphene surface provides a rapid, highly selective change in the G-FET source-drain current and the Dirac point. The instrument may be label-free since there is no requirement to modify or label the target. The instrument may be a portable hand-held instrument capable of detecting drug and/or drug metabolites at actionable levels in wastewater in less than 30 minutes. The instrument may be used for easy and frequent field sampling further upstream in community sewer lines which can be accessed by removing manhole covers. The instrument may include data storage and wireless communications for the real-time dissemination of actionable data.

Some desirable features, attributes and/or advantages of one or more embodiments of the present invention may include one or more of the following:
The invention provides the first aptamer modified G-FET sensor for rapid detection of drugs (e.g., opioids) and/or drug metabolites in wastewater.
The invention utilizes aptamer-modified G-FETs that can be reproducibly mass-made with established fabrication technologies.
The present method provides fast (<30 minutes sample-to result) test times.
The present device may be a handheld, user-friendly, smart, portable instrument at low cost (estimated user cost of $2,000 (instrument) and < $25 per test with disposable test chips compared to HPLC-MS/MS at >$200/sample).
The flat micro-scale, single-layer graphene on a rigid silicon substrate provides a sturdy surface for depositing the contact electrodes by conventional masking techniques.
The invention involves successful identification and characterization of aptamers with strong affinity to noroxycodone, norfentanyl, and EDDP.
The invention represents the first demonstration of a G-FET aptasensor for opioid detection in buffer and wastewater samples.
The invention represents a demonstration of the stability and reproducibility of opioid drug metabolite targets (noroxycodone, norfentanyl and EDDP).
The invention represents the first high-performance multiplexing capabilities, all electrical and rapid (-30 minutes) detection, with auto-calibration, of opioid markers.
Linear aptamer-based graphene field effect transistor response (change in Dirac point) occurs in buffer and wastewater samples for all selected opioid metabolites (noroxycodone, norfentanyl, and EDDP).
The present invention may be used for detecting environmental levels of opioid metabolites with a limit of detection (LOD) value of 3 pg/mL in buffer and 27 pg/mL in wastewater samples (making it the most sensitive method to date).
The selected, optimized, and validated aptamers had strong binding constant (K_{d}) values of 0.9 nM-42.6 nM using Microscale Thermophoresis (MST) and showed no affinity for other drug metabolites (no false positives).

The following examples are given for illustrative purposes only and are not meant to be a limitation on the invention described herein or on the claims appended hereto.

### Example 1: Selection and Validation of Aptamers via Spectroscopic Characterization

Three different opioid metabolites were selected, namely, (i) noroxycodone (NX), which is a metabolite of oxycodone (a semisynthetic opioid); (ii) 2-ethylidene-1,5-dimethyl-3,3-diphenyl-pyrrolidine (EDDP), which is a metabolite of methadone (a fully synthetic opioid), and (iii) norfentanyl (NF), which is a metabolite of fentanyl (a synthetic opioid). The following 5’-amine-aptamer-3’ aptamers were obtained from Base Pair Biotechnologies, Inc., Pearland, TX, for selective binding to the aforementioned opioid metabolites: (i) CFA0079-GP5-25, AKA-H4LFD (for binding to noroxycodone); (ii) CFA0661-GP5-25 (for binding to EDDP); and (iii) CFA0071-GP5-25, AKA-H6AAZ (for binding to norfentanyl). All selected aptamers were believed to have strong binding affinities to their respective opioid metabolites. Nevertheless, to verify, the nanoplasmonic assay described below was performed.

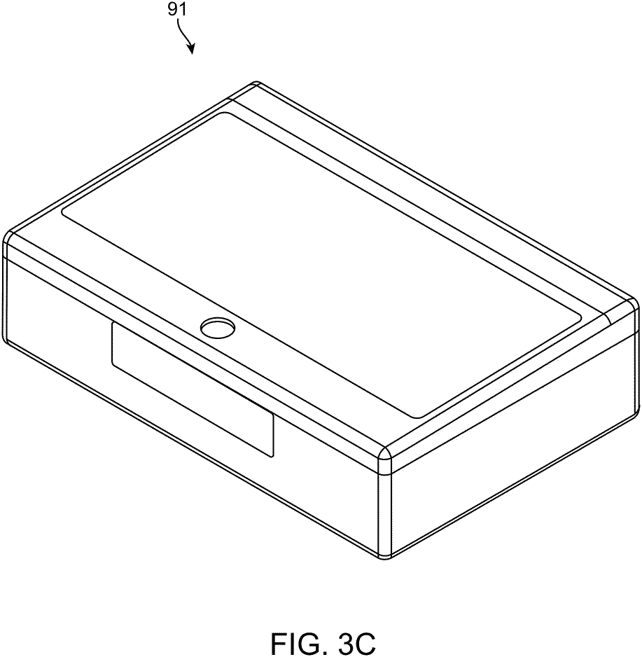

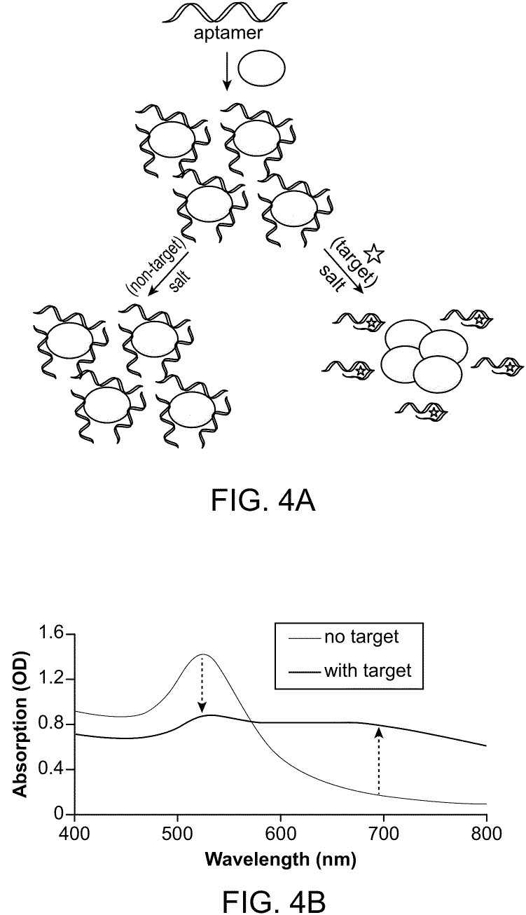

Citrate-reduced gold nanoparticles (AuNP) possess negative charge. The electrostatic repulsive forces between gold nanoparticles give them their characteristic red color (520 nm) when they are dispersed. In the presence of a negatively charged aptamer and 150 mM NaCl, a negative charge cloud protects the gold nanoparticles from any aggregation (FIG. 4A). By contrast, when an aptamer binds to a target, it leaves its associated gold nanoparticle surface, thereby reducing the inter-particle distance between the vacated gold nanoparticle and a neighboring gold nanoparticle. Salt-induced aggregation of the gold nanoparticles then takes place, resulting in a red-to-purple color transition (i.e., a transition from 520 nm to 700 nm) in less than a minute.

This simple mechanism allows one to obtain quantitative binding information by monitoring the optical density (OD) at 520 nm and the ratio of (OD₅₂₀/ OD₇₀₀) (FIG. 4B), demonstrating aptamer functionality in buffer and wastewater matrices. This procedure was used to validate the aptamers for noroxycodone, norfentanyl, and EDDP in buffer with a turnaround time of less than 15 min. This assay may also be used to determine the binding constant and selectivity of each aptamer in the relevant wastewater media for individual target drug metabolites.

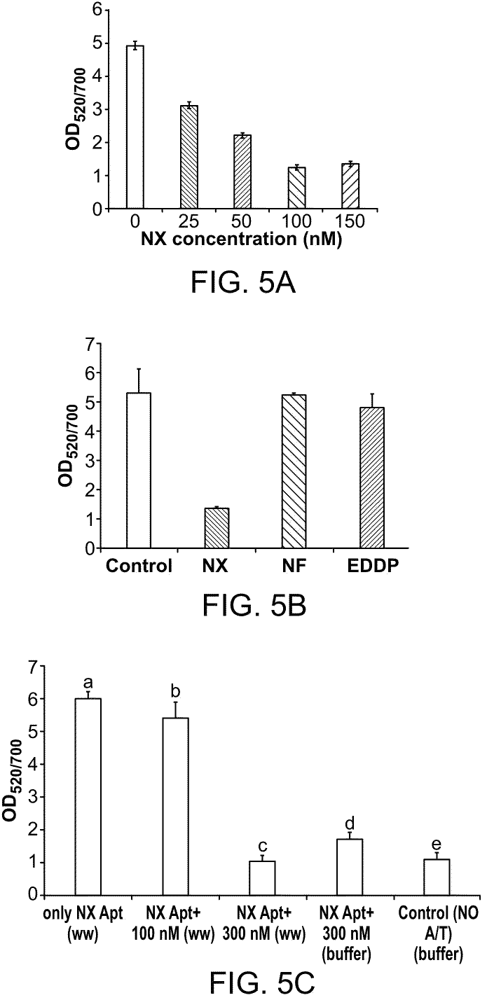

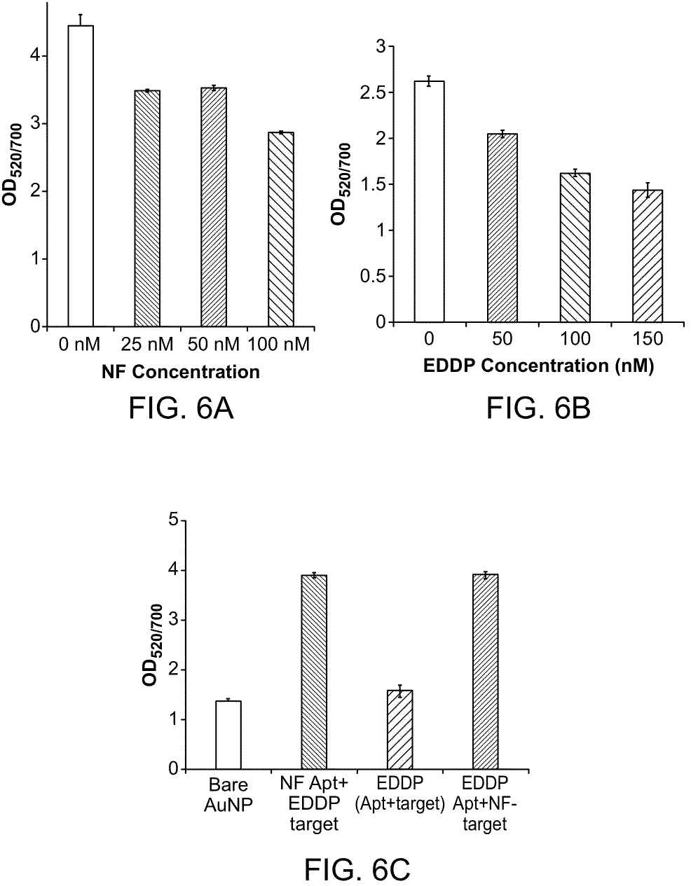

Using this colorimetric assay, sensitivity and specificity analyses of the subject aptamers for noroxycodone, norfentanyl, and EDDP were performed. FIG. 5A demonstrates a dose-dependent linear correlation between the absorbance reading (OD₅₂₀/ OD₇₀₀) and various noroxycodone levels with a limit of detection (LOD) of 6.05 nM. As expected, a more drastic color change was observed when higher dosing of noroxycodone was introduced into the buffer. These findings show that the aptamer for noroxycodone is capable of distinguishing different levels of the noroxycodone target. Similar findings were observed for the aptamers for EDDP and norfentanyl with their respective target drug analytes (FIGS. 6A through 6C). According to the specificity analysis shown in FIG. 5B, in the presence of the noroxycodone aptamer, when a non-target of norfentanyl or EDDP was introduced, the absorbance reading showed nearly no difference compared to the control (only aptamer) result. The salt-induced aggregation only takes place with the correct aptamer and noroxycodone target, and a ratio (OD₅₂₀/ OD₇₀₀) equivalent to aggregation state is measured. This data confirms the absence of any false positive or false negative detection and verifies the specificity of the chosen aptamer. (High rates of false positives and false negatives are major drawbacks for existing immunoassays that rely on antibody binding.) After sensitivity and specificity confirmation in buffer solutions, a different experimental setting was designed to detect opioid in wastewater samples compared to a buffer environment. More specifically, as seen in FIG. 5C, a distinguishable detection of noroxycodone in wastewater samples required at least 300 nM of noroxycodone as compared to 100 nM of noroxycodone when measured in buffer. So, the working condition of the aptamer:target is 1:1 in buffer (FIG. 5A) and 1:3 in wastewater samples (FIG. 5C). In summary, the functionality of all aptamers for detection of the three opioid metabolites (i.e., noroxycodone, norfentanyl, and EDDP) both in buffer and in wastewater samples (FIGS. 6A through 6C) has been validated successfully.

### Example 2: Aptamer-Based Graphene Field Effect Transistor

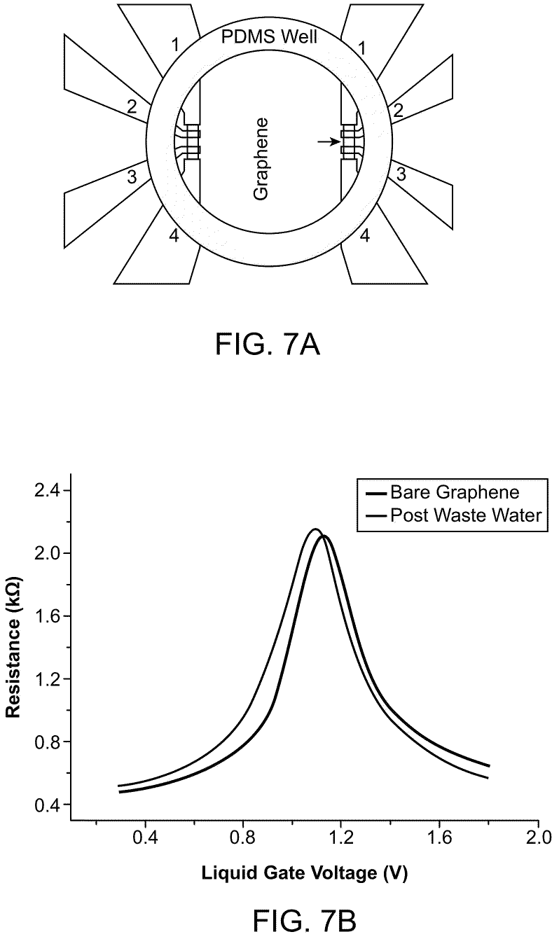

A graphene field effect transistor (G-FET) was fabricated according to a technique like that disclosed in Kumar et al., “Rapid, Multianalyte Detection of Opioid Metabolites in Wastewater,” ACS Nano, 16(3): 3704-3714 (Feb. 24, 2022), which is incorporated herein by reference. The G-FET device included a graphene film on a SiO₂/Si substrate, Au/Cr (or Pt/Ti) drain and source electrodes, and a Pt side gate. To reduce unwanted chemical reactions, the graphene device was surrounded by an Al₂O₃ layer such that only the graphene was able to be in contact with the test solution. The device was fabricated with two wells, each with two active G-FETs, as shown in FIG. 7A. To minimize the effects of the chemicals used in functionalizing the graphene, as well as to ensure maximum overlap with the active region, a poly(dimethylsiloxane) (PDMS) well was employed. PDMS (i.e., silicone) is typically produced by pouring into a mold and curing the separated wells, enabling sensing of two different analytes while multiple G-FETs in each well enhance the likelihood of attachment and robustness. Initially, four-point measurements were performed, but resistance changes far exceeded the contact resistance, so a two-point resistance design was eventually implemented. Preliminary results were obtained using a solution-gated G-FET, which resulted in higher sensitivity by significantly increasing the charge build-up on the graphene.

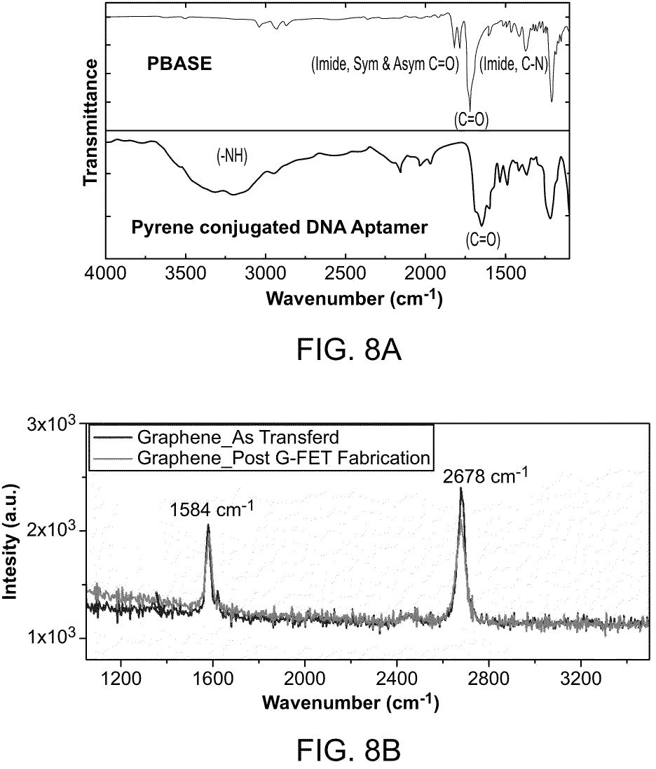

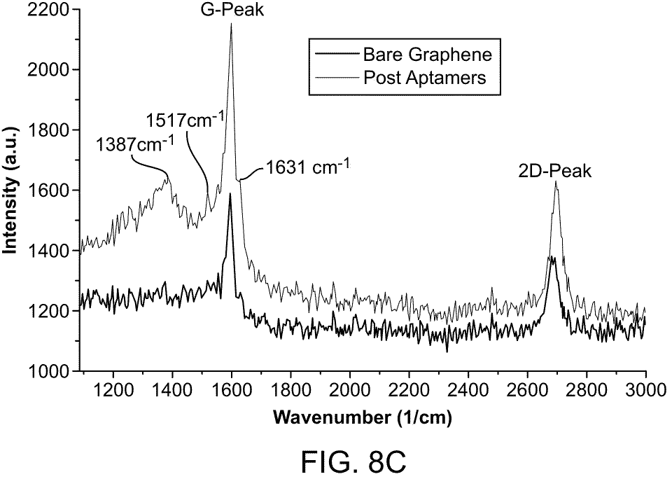

The G-FET was initially treated with pyrenebutyric acid N-hydroxysuccunumide ester (PBASE) to act as a heterobifunctional linker molecule, followed by incubation with the amino-modified opioid aptamer in phosphate-buffered saline, PBS (pH 7.4). Fourier-transform infrared (FTIR) spectroscopy was used to confirm the similar PBASE peak is unchanged on different modification steps on the graphene surface (see FIG. 8A). Raman spectra showing D, G and 2D peaks before and after fabrication process is shown in FIG. 8B. The G peak shows no shift and remains at 1584 cm⁻¹, signifying no doping of the graphene occurred during the fabrication process. The reduction in 2D peak is due to non-charge carrying contaminants/defects introduced during fabrication. This could arise from chemicals and possible contaminants on graphene, electrodes, and Si/SiO₂ substrate. No emergence of D peak observed post fabrication process implies no additional disorder induced during fabrication process. FIG. 8C shows Raman spectra of graphene over SiO₂/Si substrates before and after aptamers’ functionalization. The emergence of three new peaks at 1387, 1517, and 1631 cm-¹ confirmed the attachments of aptamers over graphene. The functionalized G-FETs were then tested with varying amounts of drugs in both PBS and wastewater samples.

Additional G-FETs were designed and fabricated with strips of graphene of various widths. Initial tests of electrical performance and uniformity were performed. These initial devices had PDMS wells attached. Chemical vapor deposition (CVD) graphene was obtained, where the CVD process was optimized for the needed grain size and doping levels. The graphene films were tested for uniformity using micro-Raman spectroscopy. Each device was put through a gate voltage sweep to measure the as-fabricated maximum resistance and to determine the mobility. By dividing the AC voltage by the AC bias current, Dirac points and resistance across the channel contacts were obtained. A number of device designs were attempted, with the final one chosen to maximize the reliability of fabrication and sensing area while minimizing extrinsic contributions. In a final design, a switch was made to using platinum bottom contacts with the graphene transferred on top. This minimized the number of fabrication steps and enhanced the protection of the contacts. In addition, an on-chip gate contact was added to each well. Finally, the chip was scaled up to four wells, each well with 5 G-FET devices, all measured independently and nearly simultaneously. As with the initial design, the entire chip was covered with an Al₂O₃ protective layer to prevent shorting and biofouling, with small windows opened only on the active region. This methodology ensured that treating with raw wastewater did not adversely affect the G-FETs (see FIG. 7B).

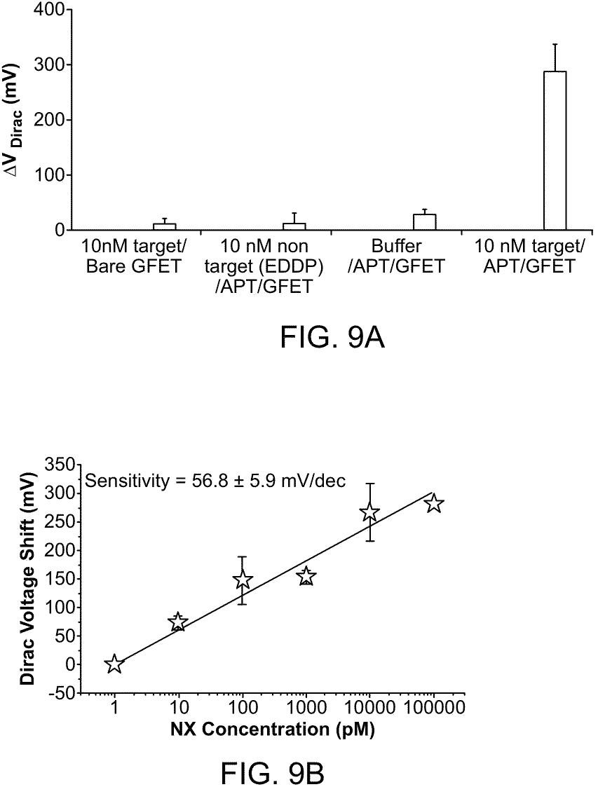

Noroxycodone, norfentanyl, and EDDP were detected with the above-described aptamer-based G-FETs in buffer and wastewater solutions at pg/mL levels. In FIG. 9A, using a noroxycodone-specific aptamer, the bar graph shows no difference between bare G-FET, APT/G-FET, and EDDP/APT/G-FET. The highest Dirac shift was only observed when the noroxycodone target was introduced. Before wastewater testing, it was confirmed that the G-FET platform can detect any drug metabolite in buffer solutions at low pg/mL (LOD= 3 pg/mL) with no false positive or negative signals. A dose-dependent calibration curve for a broad range of noroxycodone concentrations confirmed its potential dynamic range of sensitivity (FIG. 9B). Similar findings were observed for other drug metabolites EDDP and NF (data not shown). Overall, this data confirms achievement towards anticipated sensitivity/specificity milestones.

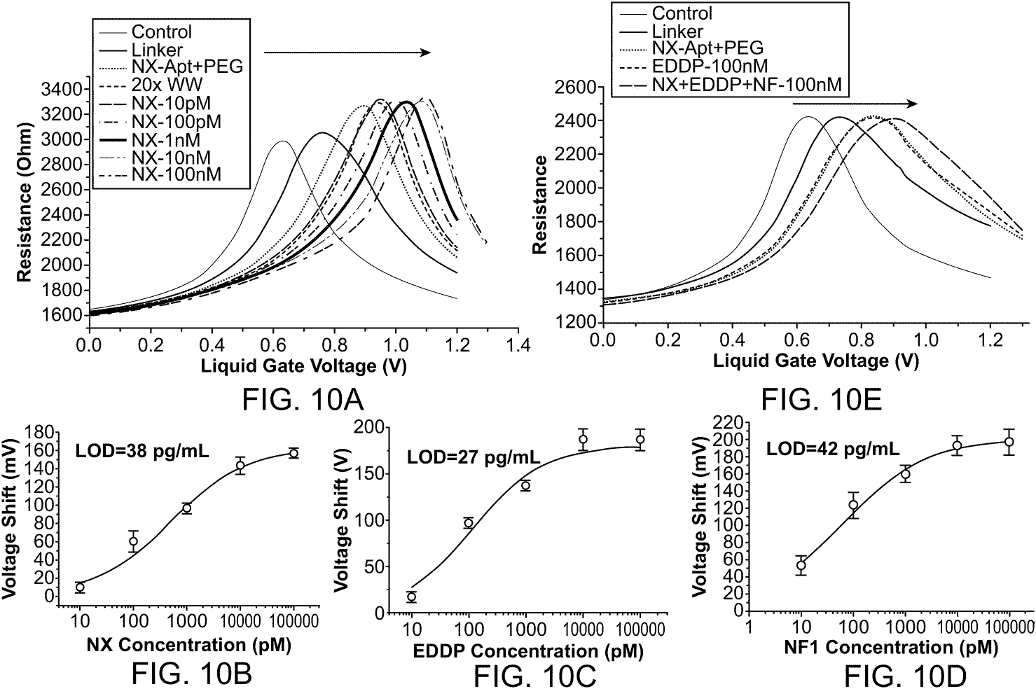

Detection limit of opioids was initially affected in wastewater due to interference caused by the presence of several other contaminants. This issue was resolved by mixing both end amine terminated polyethylene glycol (PEG) with aptamers. PEG helps to minimize the unspecific interaction with the graphene surface and reduces the Debye screening effect, which resulted in significant improvement in LOD. In FIG. 10A, a Dirac shift confirmed dose-dependent (10 pM to 100 nM) noroxycodone detection in wastewater samples. FIG. 10B shows the calibration curve. A similar correlation was observed for EDDP and norfentanyl detection in wastewater samples, where norfentanyl shows the highest voltage shift at 10 pM concentration (FIGS. 10C and 10D). The 3-sigma rule based statistical LOD for noroxycodone is 38 pg/mL, 27 pg/mL for EDDP, and 42 pg/mL for norfentanyl in 20× diluted wastewater samples. This sensitivity can be improved even lower than 10 pg/mL (in buffer, LOD is 3 pg/mL) if additional dilution steps are considered or if dielectrophoresis is employed to improve attachment. In FIG. 10E, no difference can be seen in Dirac point shift between noroxycodone-aptamer with PEG vs. noroxycodone-aptamer with EDDP (100 nM). By contrast, in the presence of the noroxycodone aptamer, when a cocktail mixture of noroxycodone, norfentanyl, and EDDP was introduced, a distinguishable Dirac shift was observed from non-target or controls. Overall, FIGS. 10A through 10E indicate that the present G-FET testing platform is capable enough in detecting illicit drug metabolites (such as noroxycodone, norfentanyl, and EDDP) in challenging wastewater samples. To improve sensitivity performance, a 0.2 µm filtration and 20× dilution with 1× PBS was used.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A method for detecting one or more drugs and/or drug metabolites of interest in a liquid sample, the method comprising the steps of:
(a) providing a device, the device comprising a graphene field effect transistor and a first aptamer coupled to the graphene field effect transistor, the first aptamer being selective for a first drug or drug metabolite of interest;
(b) exposing a liquid sample to the first aptamer of the device;
(c) then, applying a liquid gate voltage to the device and measuring the resultant resistance; and
(d) comparing the resultant resistance to appropriate standards to determine the presence and/or quantity of the first drug or drug metabolite of interest.

2. The method as claimed in claim 1 wherein step (c) comprises sweeping the liquid gate voltage to obtain a resistance versus liquid gate voltage plot for the device.

3. The method as claimed in claim 2 wherein step (d) comprises comparing a Dirac voltage shift for the device to appropriate standards.

4. The method as claimed in claim 1 wherein the first drug or drug metabolite is selected from the group consisting of oxycodone, noroxycodone, fentanyl, norfentanyl, morphine, and 2-ethylidene-1,5-dimethyl-3,3-diphenyl-pyrrolidine.

5. The method as claimed in claim 1 wherein the liquid sample is a wastewater sample.

6. The method as claimed in claim 1 wherein the one or more drugs and/or drug metabolites of interest is exactly one drug or drug metabolite.

7. A method for detecting one or more drug and/or drug metabolites of interest in a liquid sample, the method comprising the steps of:
(a) providing a device, the device comprising a graphene field effect transistor, the graphene field effect transistor having a first well and a second well, the device further comprising a first aptamer and a second aptamer, the first aptamer being selective for a first drug or drug metabolite of interest and being coupled to the graphene field effect transistor in a first well, the second aptamer being selective for a second drug or drug metabolite of interest and being coupled to the graphene field effect transistor in a second well, the second drug or drug metabolite of interest being different than the first drug or drug metabolite of interest;
(b) exposing a liquid sample to the first aptamer and the second aptamer of the device;
(c) then, applying a liquid gate voltage to each of the first well and the second well of the device and measuring the resultant resistance; and
(d) comparing the resultant resistance from each of the first well and the second well to appropriate standards to determine the presence and/or quantity of the first drug or drug metabolite of interest and the second drug or drug metabolite of interest.

8. The method as claimed in claim 7 wherein step (c) comprises sweeping the liquid gate voltage in each of the first well and the second well to obtain first and second resistance versus liquid gate voltage plots, respectively, for the device.

9. The method as claimed in claim 8 wherein step (d) comprises comparing a Dirac voltage shift for each of the first and second wells to appropriate standards.

10. The method as claimed in claim 7 wherein the one or more drugs or drug metabolites of interest are selected from the group consisting of oxycodone, noroxycodone, fentanyl, norfentanyl, morphine, and 2-ethylidene-1,5-dimethyl-3,3-diphenyl-pyrrolidine.

11. The method as claimed in claim 7 wherein the liquid sample is a wastewater sample.

12. The method as claimed in claim 7 wherein the device further comprises a third well and a third aptamer, the third aptamer being selective for a third drug or drug metabolite of interest and being coupled to the graphene field effect transistor in the third well, the third drug or drug metabolite of interest being different than the first and second drugs or drug metabolites of interest, and wherein the method further comprises exposing the liquid sample to the third aptamer of the device; then, applying a liquid gate voltage to the third well of the device and measuring the resultant resistance; and comparing the resultant resistance from the third well to appropriate standards to determine the presence and/or quantity of the third drug or drug metabolite of interest.

13. The method as claimed in claim 13 wherein the device further comprises a fourth well and a fourth aptamer, the fourth aptamer being selective for a fourth drug or drug metabolite of interest and being coupled to the graphene field effect transistor in the fourth well, the fourth drug or drug metabolite of interest being different than the first, second and third drugs or drug metabolites of interest, and wherein the method further comprises exposing the liquid sample to the fourth aptamer of the device; then, applying a liquid gate voltage to the fourth well of the device and measuring the resultant resistance; and comparing the resultant resistance from the fourth well to appropriate standards to determine the presence and/or quantity of the fourth drug or drug metabolite of interest.

14. A device for use in detecting one or more drugs and/or drug metabolites of interest in a liquid sample, the device comprising:
(a) a graphene field effect transistor, the graphene field effect transistor comprising a first well; and
(b) a first aptamer, the first aptamer being coupled to the graphene field effect transistor in the first well, the first aptamer being selective for a first drug or drug metabolite of interest.

15. The device as claimed in claim 14 wherein the first aptamer is selective for a drug or drug metabolite selected from the group consisting of oxycodone, noroxycodone, fentanyl, norfentanyl, morphine, and 2-ethylidene-1,5-dimethyl-3,3-diphenyl-pyrrolidine.

16. The device as claimed in claim 14 wherein the first aptamer is coupled to the graphene field effect transistor using a linker molecule.

17. The device as claimed in claim 16 wherein the linker molecule is pyrenebutyric acid N-hydroxysuccunumide ester.

18. The device as claimed in claim 14 wherein the graphene field effect transistor further comprises a second well and wherein the device further comprises a second aptamer, the second aptamer being coupled to the graphene field effect transistor in the second well, the second aptamer being selective for a second drug or drug metabolite of interest, the second drug or drug metabolite of interest being different than the first drug or drug metabolite of interest.

19. A system for detecting one or more drug and/or drug metabolites in a liquid sample, the system comprising the device of claim 13, a voltage sweep generator for applying a voltage sweep to the graphene field effect transistor, and a reader/analyzer for measuring the resultant resistance and comparing the resultant resistance to appropriate standards.
